(19) United States
(12) Patent Application Publication
(10) Pub. No.: US 2011/0124899 A1
(43) Pub. Date: May 26, 2011

(54) METHOD FOR PREPARING COMBRETASTATIN

(75) Inventors: Marc FREDERIC, Bretigny Sur Orge (FR); Sylviane LUTZ, Emont (FR); Joel MALPART, Olivet (FR); Philippe MASSON, Lisses (FR); Stephane MUTTI, Le Perreux sur Marne (FR)

(73) Assignee: SANOFI-AVENTIS, Paris (FR)

(21) Appl. No.: 12/869,997

(22) Filed: Aug. 27, 2010

Related U.S. Application Data
(63) Continuation of application No. PCT/FR2009/000215, filed on Feb. 27, 2009.

(30) Foreign Application Priority Data
Feb. 28, 2008 (FR) 0801092

(51) Int. Cl. C07F 9/6571; C07C 231/00; C07B 57/00

(52) U.S. Cl. 558/73; 564/194; 564/304

(57) ABSTRACT

The Invention relates to a method for preparing a combretastatin (A): formula (I) in the form of a base or of an addition salt with an acid, which comprises coupling, in the presence of a base and of T3P, the salt of the (Z)-amino compound of formula (II) with a doubly protected L-serine derivative of formula (III) in which PG denotes a group protecting the amine function, so as to obtain the compound of formula (Z)-(Ib): formula (IV), then deprotecting and opening the ring of (Z)-(Ib) in the presence of an acid, so as to obtain the combretastatin (A) in the form of a salt; and, optionally, adding a base, so as to obtain the combretastatin (A) in the form of a base, the salt of the (Z)-amino compound having been obtained by enrichment of the salt of the amino compound of formula (V) in (Z) isomer.

The present application relates to a method for preparing a combretastatin (A):

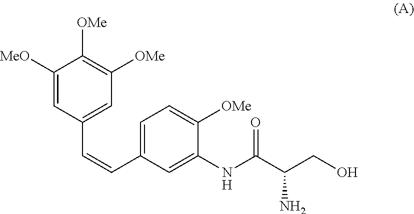

in the form of a base or of an addition salt with an acid.

### PRIOR ART

U.S. Pat. No. 6,759,555 describes a method for preparing a combretastatin of formula:

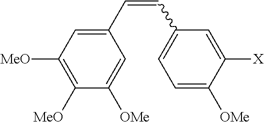

in which X represents —NH₂ or one of the following two groups:

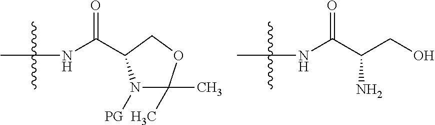

PG denoting a group protecting the amine function.
Bioorg. Med. Chem. 2000, 8, 2417-2425 and US 2003/0220404 also describe methods for preparing combretastatins.
J. Pept. Res. 1999, 54(1), 54-65 compares acid activators in the formation of peptide bonds and concludes that TDBTU is the best.
Bioorg. Med. Chem. 2006, 14, 3231-3244 describes the preparation of combretastatin of formula (A) with coupling using, as acid activator, DCC, HOBt-H₂O (step e, compound 10).
Chem. Commun. 1999, 1847-1848 describes T3P in the preparation of amide bonds; however, T3P is described as producing more epimerization and resulting in poorer yields than HAPyU (see table 3).

The method which is the subject of claim 1 is neither described nor suggested in these documents. Similarly, neither the enrichment of the amino compound salt using benzyl alcohol and acetonitrile nor the coupling using T3P is described.

### THE TECHNICAL PROBLEM

Combretastatins or stilbene derivatives exhibit a strong cytotoxic activity and as a result can be used as anticancer agents. However, it is the (Z) isomers which exhibit the strongest cytotoxic activity. These compounds are in particular described in U.S. Pat. No. 5,731,353, U.S. Pat. No. 5,561,122 or U.S. Pat. No. 6,759,555. The Applicant has improved the method for preparing combretastatin (A).

### BRIEF DESCRIPTION OF THE INVENTION

The invention relates to a method for preparing a combretastatin (A):

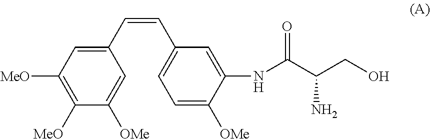

in the form of a base or of an addition salt with an acid, consisting in coupling, in the presence of a base and of T3P of formula (III):

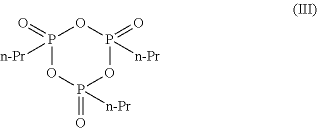

the (Z)-amino compound

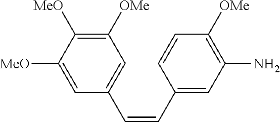

or the salt of the (Z)-amino compound

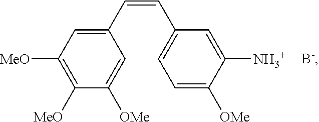

B⁻ denoting a counteranion, with a doubly protected L-serine derivative of formula

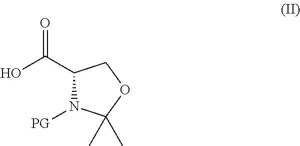

in which PG denotes a group protecting the amine function, so as to obtain the compound of formula (Z)-(Ib):

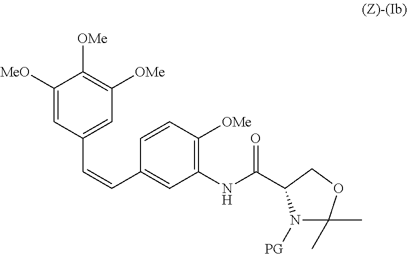

and then in deprotecting and opening the ring of (Z)-(Ib) in the presence of an acid, so as to obtain the combretastatin (A) in the form of a salt (—NH₃⁺) and, optionally, in adding a base so as to obtain the combretastatin (A) in the form of a base (—NH₂),
the salt of the (Z)-amino compound having been obtained by enrichment of the salt of the amino compound of formula:

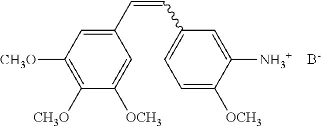

in (Z) isomer, B⁻ denoting a counteranion, consisting:
in adding benzyl alcohol to a suspension of a mixture of the salts of the (Z)- and (E)-amino compounds in acetonitrile, then
in mechanically separating the salt of the amino compound enriched in (Z) isomer.

The invention also relates to the enrichment of the salt of the amino compound of formula

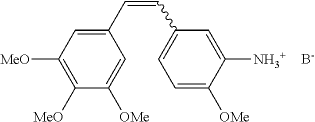

in (Z) isomer, B⁻ denoting a counteranion, consisting:
in adding benzyl alcohol to a suspension of a mixture of the salts of the (Z)- and (E)-amino compounds in acetonitrile, and
in mechanically separating the salt of the amino compound enriched in (Z) isomer.

The invention also relates to the use of T3P of formula (III)

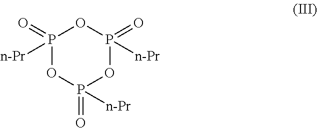

for coupling, in the presence of a base, the (Z)-amino compound or the salt of the (Z)-amino compound with the doubly protected L-serine derivative.

The acetonitrile/salts of the (Z)- and (E)-amino compounds proportion, expressed by weight, is between 5 and 17, preferably between 10 and 12. The temperature at which the enrichment is carried out is preferably between 20 and 70° C. The benzyl alcohol/salts of the (Z)- and (E)-amino compounds proportion, expressed by weight, is between 1 and 4, preferably between 2 and 3.

### DETAILED DESCRIPTION OF THE INVENTION

Scheme 1 below describes the reaction steps (i) to (iv) of a method for preparing combretastatin (A):

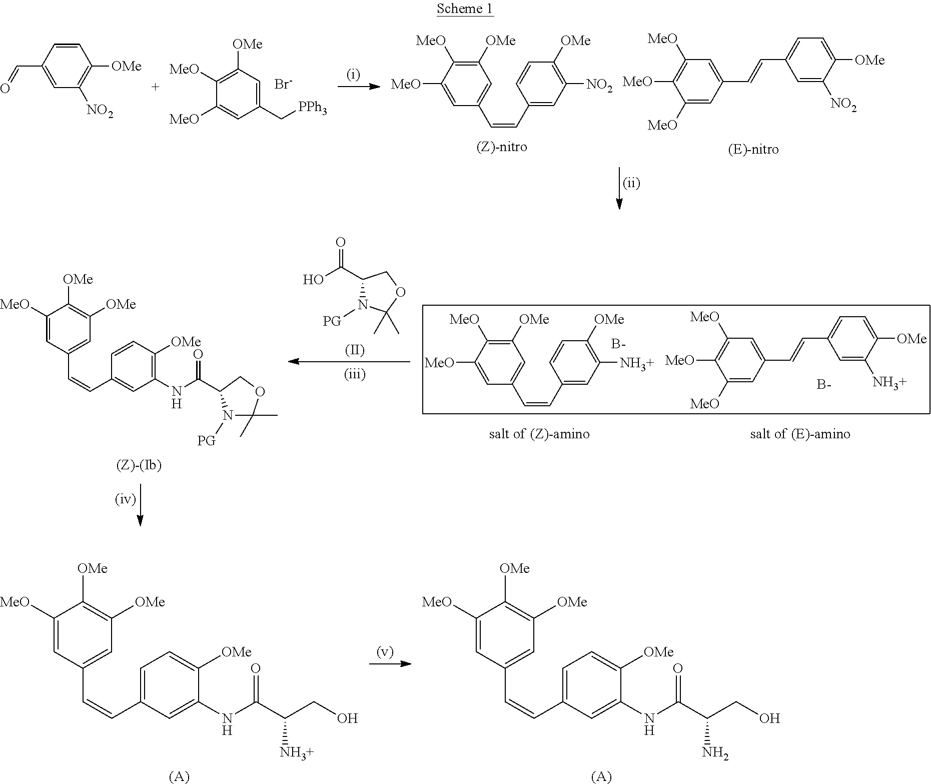

step (i): Wittig reaction between nitromethoxybenzaldehyde and trimethoxybenzylphosphonium bromide or chloride in the presence of a base, producing the salt of the nitro compound, which is in the form of a mixture of the two (Z) and (E) isomers of 2-methoxy-5-[2-(3,4,5-trimethoxyphenyl)vinyl]nitrobenzene ((Z)- and (E)-nitro);
step (ii): reduction of the mixture, resulting in a mixture of the (Z)- and (E)-amino compounds which are converted to salts (B⁻ denotes a counteranion, for example Cl⁻ or SO₄²), followed by a step of separating the (Z)-amino compound;
step (iii): coupling of the salt of the (Z)-amino compound with an L-serine derivative doubly
   protected on the —OH and amino functions (compound of formula (II)), resulting in the compound (Z)-(Ib).

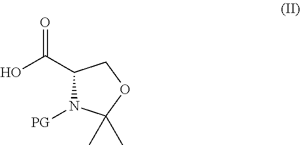

PG denotes a group protecting the amine function: it may, for example, be tert-butoxycarbonyl (BOC), benzyloxycarbonyl (CBZ) or 9-fluorenylmethyloxycarbonyl (FMOC);
steps (iv) and (v): deprotection and opening of the ring, resulting in combretastatin (A) in salified or nonsalified form.

Step (i) is described in U.S. Pat. No. 5,731,353 and also in Bioorg. Med. Chem. 2000, 8, 2417-2425 (Scheme 1). The reaction is carried out in an organic solvent such as, for example, an aromatic solvent (for example, toluene), in the presence of a base, preferably a strong base, such as MeONa or NaH. The (Z)/(E) ratio is of the order of 75/25.

The reduction step (ii) described in U.S. Pat. No. 6,759,555 is carried out in the presence of excess iron (more than 2 equivalents relative to (Ia)). Reduction in the presence of zinc, as described in U.S. Pat. No. 5,525,632 (Example 2, step 2) is also possible, but it is not complete (yield=49.3%) and it results, in addition, in the formation of a large amount of “azo” by-product. The (Z)-amino compound of sufficient purity is subsequently obtained by means of one or more complicated separation(s). The separation which follows the reduction is carried out by successive crystallizations. A 1^{st} crystallization makes it possible to remove the (E)-amino compound, and then a 2^{nd} crystallization makes it possible to isolate the (Z)-amino compound (see U.S. Pat. No. 6,759,555, Example 1).

The coupling of step (iii) is advantageously carried out in the presence of an acid activator such as, for example, EDCl (1-(3-dimethylaminopropyl)-3-ethylcarbodiimide chloride), DCC (dicyclohexylcarbodiimide), TOTU (O-[ethoxycarbonyl]cyanomethyleneamino)-N,N,N′,N′-tetramethyluronium tetrafluoroborate), HBTU (O-benzotriazol-1-yl-N,N,N′,N′-tetramethyluronium hexafluorophosphate), PivCl (pivaloyl chloride) or N,N-carbonyldiimidazole. The term “acid activator” (“coupling agent”) is used to denote a compound of which the function is to activate the acid function —COOH in order to promote the formation of a peptide bond. For more details on acid activators, reference may be made to the review ChemFiles Vol. 7, No. 2, page 3 published by the company Aldrich Chemical or else to Tetrahedron report No. 672, 2004, 60, 2447-2467, “Recent development of peptide coupling reagents in organic synthesis”. The review Tetrahedron 2005, 61, 10827-10852, discloses that many acid activators are available.

Step (iv) is carried out in the presence of an acid in order to promote ring opening and to obtain combretastatin (A) in the form of a salt (—NH₃⁺). It involves a deprotection/ring opening. Hydrochloric acid, for example in the form of a methanolic solution, is advantageously used, in the presence of BOC, and the hydrochloride is obtained. Combretastatin (A) in the form of a base is obtained by neutralizing the salt using a base, for example sodium hydroxide (cf. Example 1-step (iii)).

### Method According to the Invention

The method of the present invention reiterates the same steps of Scheme 1, but steps (ii) and (iii) have been improved. In fact, in the invention, during step (ii), the (Z) isomer of the salt of the amino compound is obtained by means of the method consisting:
in adding benzyl alcohol to a suspension of a mixture of the salts of the (Z)- and (E)-amino compounds in acetonitrile, and
in mechanically separating the salt of the amino compound enriched in (Z) isomer.

The (Z) isomer is therefore obtained by “enrichment”, this term meaning that, at the end of the two steps of the method described above, the proportion of (Z) isomer is increased relative to the (E) isomer; one can also speak of a method for separating the (Z) isomer from a mixture of the (Z) and (E) isomers. Compared to a (re)crystallization, enrichment has the advantage of being both direct and simple to implement. It makes it possible to obtain the amino compound enriched in (Z) isomer with a low content of residual (E) isomer (which can be up to <1 mol %; cf. Example 1, where the purity of the product is 99.93% with respect to (Z) and 0.07% with respect to (E)). The mechanical separation may, for example, be a filtration or a centrifugation. At the end of the mechanical separation, the salt of the (Z) isomer can be optionally washed and dried.

The suspension preferably has an acetonitrile/salts of the (Z)- and (E)-amino compounds proportion, expressed by weight, of between 5 and 17, preferably between 10 and 12 (i.e. the weight of acetonitrile in the suspension is between 5 and 17× the weight of the (Z)- and (E)-amino compounds).

The amount of benzyl alcohol which is added is preferably such that the benzyl alcohol/salts of the (Z)- and (E)-amino compounds proportion, expressed by weight, is between 1 and 4, preferably between 2 and 3 (i.e. the weight of benzyl alcohol added is between 1 and 4× the weight of the (Z)- and (E)-amino compounds). This proportion makes it possible to maintain a high (Z)/(E) ratio for the final product. The function of the benzyl alcohol is to preferentially dissolve the salt of the (E)-amino compound.

The enrichment is preferably carried out at a temperature of between 20 and 70° C., advantageously between 30 and 70° C., preferably between 35 and 65° C. Above 70° C., the amino compound begins to slowly decompose.

A preferred method for preparing the suspension is described hereinafter. The (Z)- and (E)-nitro compounds are reduced using sodium dithionite (Na₂S₂O₄) in a solvent which may be a mixture of water and alcohol, for example a water/methanol mixture. After the reduction, a strong acid (for example, HCl or H₂SO₄) is introduced into the reaction medium, and said acid reacts with the reaction intermediates and also with the dithionite and disulphite residues. A mixture of the salts of the (Z)- and (E)-amino compounds (for example, hydrochloride or sulphate) is then obtained. A strong base is then added so as to obtain the free bases (—NH₃⁺ [symbol] —NH₂) which are extracted with an organic solvent, for example a chlorinated solvent such as dichloromethane (DCM). A strong acid in an alcohol is added to the organic phase, and then the alcohol is displaced with acetonitrile so as to obtain a suspension of the salts of the (Z)- and (E)-amino compounds in acetonitrile. The strong acid may be HCl or H₂SO₄ (B⁻=Cl or SO₄). The solvent displacement may be carried out by adding the acetonitrile after having more or less completely eliminated the alcohol under vacuum. The addition of acetonitrile may also be carried out concomitantly with the elimination of the alcohol under vacuum. The alcohol is preferably a light alcohol such as methanol or ethanol.

For step (iii), the coupling between the salt of the (Z)-amino compound and the doubly protected L-serine derivative of formula (II) is carried out in an organic solvent using propanephosphonic acid anhydride (T3P) as acid activator in the presence of a base. The function of the base is to trap the acid species and to shift the salt towards the free base. The amount of base added is between 2 and 3 eq. relative to the salt of the (Z)-amino compound (see Example 1, where 2.7 eq. are used). It is also possible to directly couple the (Z)-amino compound with the compound of formula (II) in the presence of T3P and of a base; in this case, the proportion of base added is smaller, between 1 and 2 eq. (approximately 1 eq. less than when starting from the salt).

The base may be a tertiary amine such as, for example, triethylamine (TEA), diisopropylethylamine (DIEA), N-methylmorpholine (NMM) or methylpiperidine. The organic solvent may be dichloromethane (DCM), toluene, methyl isobutyl ketone (MIBK), ethyl acetate, acetonitrile, tetrahydrofuran (THF), Me-tetrahydrofuran (Me-THF) or cyclopentyl methyl ether.

T3P has the formula:

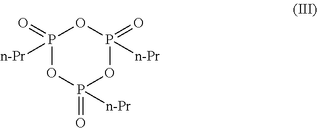

The advantage of using T3P rather than another acid activator is that the by-products of T3P can be readily eliminated (by-products all soluble in water) and that it is an inexpensive activator. In addition, the reaction may be carried out in the presence of the (Z)-amino compound (in salt or base form) and of the compound (II), brought together according to a “one pot” process: the (Z)-amino compound (in the form of a salt or of a base) and the compound (II) are therefore reacted together, in the same container, in the presence of T3P and of a base. This is not the case of all coupling reagents because some, such as PivCl (pivaloyl chloride), require, for the same reaction, prior activation of the acid function of the compound (II) before being able to add the salt of the (Z)-amino compound.

Finally, we have noted that T3P does not result in epimerization of the asymmetric centre, thereby making it possible to obtain combretastatin (A) with a good purity and a good yield. It has also been noted that T3P makes it possible to obtain a good yield in terms of coupling product (see table III).

The coupling reaction is generally carried out at a temperature of between 5° C. and 70° C., for example at the reflux of DCM. The proportion of T3P relative to the (Z)-amino compound is between 1 and 2 eq, preferably between 1.5 and 1.8 eq.

### EXAMPLES

### Example 1

### According to the Invention

### Step (i):

A solution of sodium methoxide (5.66 kg, 29.34 mol) is run into a mixture, at a temperature of 5-10° C., comprising toluene (91.1 litres), trimethoxybenzylphosphonium bromide (15.35 kg, 29.33 mol) and 4-methoxy-3-nitrobenzaldehyde (5.06 kg, 27.93 mol). At the end of the reaction, 0.32 litre (5.59 mol) of acetic acid is run in. After the medium has been maintained at 20° C., it is filtered. The cake is washed with toluene (11.1 litres). The filtrates are washed several times with water (20.2 litres) and then concentrated under vacuum. Isopropyl alcohol (87.6 litres) is then introduced and the medium is concentrated and then cooled. The suspension is then filtered at 10° C. The isolated product is dried under vacuum (6.46 kg, 52.2%). The purity of the isolated product is of the order of 78% with respect to (Z) and 22% with respect to (E).

### Step (ii):

Water (80 ml), at 50° C., is run into a medium comprising methanol (100 ml), the (Z)- and (E)-nitro compounds to be reduced (20 g, 0.058 mol) and sodium dithionite (36.8 g, 0.179 mol, 3.1 eq). Once the reaction is complete (left for a period of 1 h at 50° C.), hydrochloric acid (36.3 ml, 0.406 mol) is added. The treatment is carried out by running in water (70 ml) and DCM (80 ml), and then sodium hydroxide (lye 30.5%, 10N) to pH=7. The aqueous phase is eliminated after reextraction with DCM (20 ml) and then the DCM phase is concentrated under vacuum (approximately 200 mbar at 35° C.) and replaced with acetonitrile (160 ml); the change in solvent from DCM to acetonitrile is carried out under reduced pressure so as to keep a reaction volume of approximately 200 ml. Methanolic hydrochloric acid (27 ml, 0.081 mol) is run in and then the solvent is evaporated off under reduced pressure (90 mbar) so as to perform the solvent change from methanol-acetonitrile to acetonitrile at a constant volume of approximately 233 ml. At the end of the solvent change, the total volume (solvent+organic compounds) is adjusted to 280 ml.

A suspension is then obtained in the form of a white broth. The benzyl alcohol (46 ml) is then added to the suspension at 45±3° C. After cooling to 25° C., the medium is then filtered and washed with acetonitrile (30 ml) and benzyl alcohol (3.3 ml). The isolated product is then dried under vacuum (11.7 g, 74.4%). This product has a purity, determined by HPLC, of 99.93% with respect to (Z) and 0.07% with respect to (E). At the end of step (ii), the salt of the (Z)-amino compound has thus been obtained simply and directly, with a good purity.

Step (iii):

TEA (53.4 ml, 0.383 mol) at 20° C. and then a solution of T3P in DCM (154 g, 0.242 mol) are run into a medium comprising DCM (500 ml), the (Z)-amino compound in hydrochloride form (50 g, 0.142 mol) and the doubly protected L-serine of formula (II) with PG=BOC (L-serine-N—BOC-acetonide; 41.8 g, 0.170 mol). The medium is brought to reflux and then water (500 ml) is added. After separation by settling out and concentration, the methanolic hydrochloric acid (189.5 ml, 1.136 mol) and methanol (189.5 ml) are added. Water (300 ml) is added to the medium and the phases are then separated by settling out. Isopropyl acetate (650 ml) is added to the aqueous phase and then sodium hydroxide (115 ml, 1.150 mol) is run in at 20° C. The organic phase which has been separated by settling out and washed is concentrated under vacuum and then heated to 65° C. Methanol (35 ml) and then methanolic hydrochloric acid (47.4 ml, 0.142 mol) are added at this temperature. After cooling, and filtration, the product is isolated (49.6 g, 79.5%). The final product has a purity of 99.2%.

### Example 2

### According to the Invention

The (Z)-amino compound in hydrochloride form Z-aminostil, HCl (50.0 g), the N—BOC-acetonide (41.8 g) and 500 ml of DCM are loaded into a 1.6 litre reactor equipped with a jacket. 53.4 ml of triethylamine (2.7 eq.) are then run in at 22±3° C., followed by a solution of T3P in DCM at 50% (1.7 eq.). The mixture is stirred at the reflux of DCM for 1 hour. The mixture is then cooled to 22±3° C. and 500 ml of demineralized water are added. The mixture is left to separate by settling out and the phases are separated. The DCM phase is washed with 500 ml of an aqueous solution of sodium hydrogen phosphate at 6 weight % (30 g), and then with 500 ml of demineralized water. The DCM phase is concentrated under reduced pressure from 360 to 150 mbar, at around 40-50° C. A solution of HCl in methanol at 3 mol/l (379 ml, 8 eq.) is then run in, followed by 300 ml of demineralized water. The mixture is left to separate by settling out and the phases are separated. The DCM/methanol phase is reextracted with demineralized water (200 ml) and the phases are separated. 650 ml of isopropyl acetate and then 115 ml of a 30% sodium hydroxide solution (8.1 eq.) are added. The mixture is left to separate by settling out and the organic phase is washed with 400 ml of demineralized water. The mixture is left to separate by settling out and the phases are separated. The organic phase is then concentrated under reduced pressure from 160 to 60 mbar until 300-350 ml are obtained, and a DCM [symbol] isopropyl acetate solvent change is carried out. The mixture is then heated at 62° C., 35 ml of methanol are added, and a solution of hydrochloric acid in methanol at 3 mol/l (47.4 ml, 1 eq.) is run in. The resulting product is then left to cool to ambient temperature and the white broth is filtered. The final solid is washed.

In Examples 3-6, step (ii) is repeated with different amounts of (Z)- and (E)-nitro compounds at the start. The acetonitrile/salts of the (Z)- and (E)-amino compounds proportion is fixed at 10.8 and the benzyl alcohol/salts of the (Z)- and (E)-amino compounds proportion is, for its part, variable.

### Example 3

### According to the Invention

Water (60 ml) at 50° C. is run into a medium comprising methanol (50 ml), the (Z)- and (E)-nitro compounds to be reduced (15 g, 0.043 mol) and sodium dithionite (27.2 g, 0.133 mol). Once the reaction is complete, hydrochloric acid (26.2 ml, 0.314 mol) is added. The treatment is carried out by running in water and DCM, and then sodium hydroxide to pH=7. Methanolic hydrochloric acid (18.9 ml, 0.0586 mol) is added to the DCM phase and then the solvent is replaced with acetonitrile. A broth is then obtained. Benzyl alcohol (31 ml) is then added to the suspension at 50° C. and maintained at 65° C. for 2 hours. After cooling, the medium is then filtered and washed. The isolated product is then dried under vacuum.

### Examples 4-6

### Identical to Example 3, but with a Different Amount of Benzyl Alcohol

TABLE I

| | benzyl alcohol/ | | |
|---|---|---|---|
| | Z + E amino salts | HPLC | crude |
| | proportion by | purity of | yield of Z- |
| | weight | Z-amino | amino |
| Ex. 4 | 1.96 | 99.5% | 82.0% |
| Ex. 3 | 2.09 | 99.6% | 81.0% |
| Ex. 5 | 2.25 | 99.7% | 77.0% |
| Ex. 6 | 2.38 | 99.9% | 75.6% |

acetonitrile/Z + E amino salts proportion = 10.8

In Examples 5 and 7-9, the benzyl alcohol/salts of the (Z)- and (E)-amino compounds proportion is fixed at 2.25 and the acetonitrile/salts of the (Z)- and (E)-amino compounds proportion is variable.

TABLE II

| | acetonitrile/Z + E | HPLC |
|---|---|---|
| | amino salts | purity of |
| | proportion by | the Z- |
| | weight | amino |
| Ex. 7 | 9.10 | 99.7 |
| Ex. 5 | 10.80 | 99.9 |
| Ex. 8 | 11.88 | 98.9 |
| Ex. 9 | 12.65 | 94.5 |

benzyl alcohol/Z + E amino salts proportion = 2.25

Examples 10-14 describe results of coupling (step (iii)) using coupling agents other than T3P.

### Example 10

### (Comparative): Use of TOTU

The conditions of step (iii) are repeated, but in the presence of TOTU as acid activator (1 eq. of TOTU+5 eq. of TEA). The final yield is then only 71%.

### Example 11

### (Comparative): Use of TOTU

TEA (0.71 g, 7.0 mmol) at 5° C. and then the TOTU (0.46 g, 1.4 mmol) are run into a medium comprising DCM (10 ml), the (Z)-amino compound in hydrochloride form (0.50 g, 1.4 mmol) and the doubly protected L-serine of formula (II) with PG=BOC (L-serine-N—BOC-acetonide; 0.35 g, 1.4 mmol). The medium is maintained at 5° C. for 24 hours and then water (5 ml) is added. After separation by settling out, the organic phase is analysed by HPLC. The quantitatively determined yield of the coupling product is 50.1% and its purity is 69.3%.

### Example 12

### (Comparative): Use of BOP-Cl (bis(2-oxo-3-oxazolidinyl)phosphinic chloride

NMM (0.42 g, 4.2 mmol) at 5° C. and then the BOP-Cl (0.72 g, 2.8 mmol) are run into a medium comprising DCM (5 ml), (Z)-amino compound (0.50 g, 1.4 mmol) and the doubly protected L-serine of formula (II) with PG=BOC (L-serine-N—BOC-acetonide; 0.35 g, 1.4 mmol). The medium is maintained at 5° C. for 24 hours and then water (5 ml) is added. After separation by settling out, the organic phase is analysed by HPLC. The quantitatively determined yield of the coupling product is 29.1% and its purity is 91.6%.

### Example 13

### (Comparative): Use of PyClOP (chlorotripyrrolidinophosphonium hexafluorophosphate)

NMN (0.42 g, 4.2 mmol) at 5° C. and then the PyClOP (1.2 g, 2.8 mmol) are run into a medium comprising ethyl acetate (10 ml), the (Z)-amino compound (0.50 g, 1.4 mmol) and the doubly protected L-serine of formula (II) with PG=BOC (L-serine-N—BOC-acetonide; 0.70 g, 2.8 mmol). The medium is maintained at 5° C. for 24 hours and then water (5 ml) is added. After separation by settling out, the organic phase is analysed by HPLC. The quantitatively determined yield of the coupling product is 53.3% and its purity is 83.9%.

### Example 14

### (Comparative): Use of PyBROP (bromo-tris-pyrrolidinophosphonium hexafluorophosphate)

NMN (0.42 g, 4.2 mmol) at 25° C. and then PyBROP (0.65 g, 1.4 mmol) are run into a medium comprising Me-CN (5 ml), the (Z)-amino compound (0.50 g, 1.4 mmol) and the doubly protected L-serine of formula (II) with PG=BOC (L-serine-N—BOC-acetonide; 0.35 g, 1.4 mmol). The medium is maintained at 25° C. for 24 hours and then water (5 ml) is added. After separation by settling out, the organic phase is analysed by HPLC. The quantitatively determined yield of the coupling product is 24.9% and its purity is 75.4%.

TABLE III

| | | | quantitatively | |
|---|---|---|---|---|
| | | | determined | |
| | | | yield of the | purity |
| | coupling | | coupling | by |
| | agent | base | product (Z)-(lb) | HPLC |
| Ex. 2 | T3P | TEA - 2.7 eq. | 99.7% | — |
| Ex. 11 | TOTU | TEA - 5 eq. | 50.1% | 69.3% |
| Ex. 12 | BOP-Cl | NMN - 3 eq. | 29.1% | 91.6% |
| Ex. 13 | PyClOP | NMN - 3 eq. | 53.3% | 83.9% |
| Ex. 14 | PyBROP | NMN - 1 eq. | 24.9% | 75.4% |

It is noted that T3P makes it possible to obtain a better yield in terms of coupling product than TOTU, BOP-Cl, PyClOP or PyBROP.

1. A method for preparing a combretastatin (A): 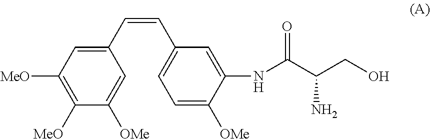
in the form of a base or of an addition salt with an acid, said method comprising the steps of:
a) coupling, in the presence of a base and of T3P of formula (III): 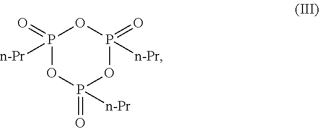
the (Z)-amino compound 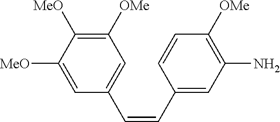
or the salt of the (Z)-amino compound 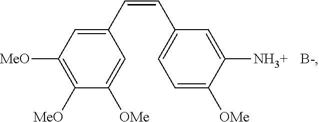
in which B⁻ denotes a counteranion, with a doubly protected L-serine derivative of formula (II): 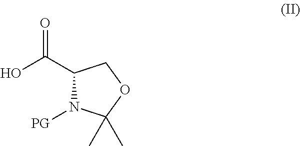
in which PG denotes a group protecting the amine function,
so as to obtain the compound of formula (Z)-(Ib): 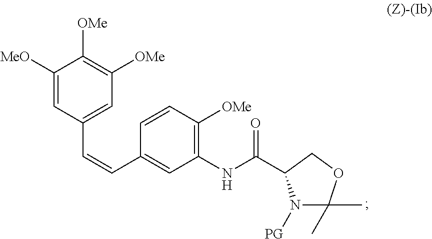
and
(b)(1) deprotecting and opening the ring of (Z)-(Ib) in the presence of an acid, so as to obtain the combretastatin of formula (A) in the form of a salt; and optionally
(b)(2) adding a base so as to obtain the combretastatin of formula (A) in the form of a base,
wherein the salt of the (Z)-amino compound is obtained by enrichment of the salt of the amino compound of formula: 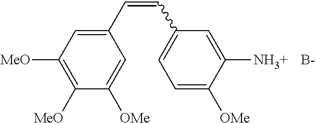
in (Z) isomer form, said enrichment comprising the steps of:
adding benzyl alcohol to a suspension of a mixture of the salts of the (Z)- and (E)-amino compounds in acetonitrile; and
mechanically separating the salt of the amino compound enriched in (Z) isomer.

2. A method for enriching the salt of the amino compound of formula: 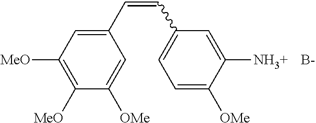
in (Z) isomer, in which B⁻ denotes a counteranion, said method comprising the steps of:
adding benzyl alcohol to a suspension of a mixture of the salts of the (Z)- and (E)-amino compounds in acetonitrile, and
mechanically separating the salt of the amino compound enriched in (Z) isomer.

3. A method according to claim 1 or 2, wherein said mechanical separation comprises filtration.

4. A method for preparing a combretastatin (A): 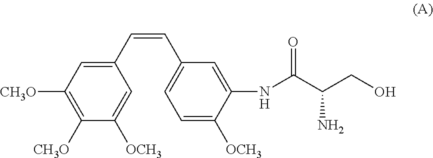
in the form of a base or of an addition salt with an acid, said method comprising the steps of:
(a) coupling, in the presence of a base and of T3P of formula (III): 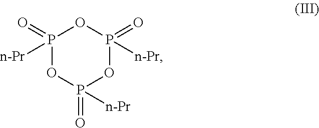
the (Z)-amino compound 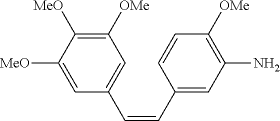
or the salt of the (Z)-amino compound 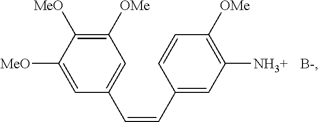
in which B denotes a counteranion,
with a doubly protected L-serine derivative of formula (II): 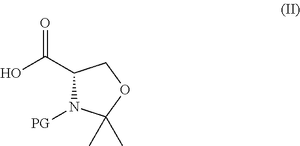
in which PG denotes a group protecting the amine function,
so as to obtain the compound of formula (Z)-(Ib): 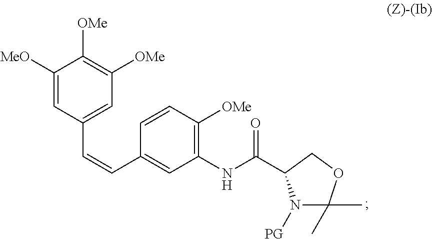
(b)(1) deprotecting and opening the ring of (Z)-(Ib) in the presence of an acid, so as to obtain the combretastatin (A) in the form of a salt; and optionally
(b)(2) adding a base so as to obtain the combretastatin (A) in the form of a base.

5. The method according to one of claim 1, 2, or 4, in which PG represents BOC, CBZ or FMOC.

6. The method according to claim 5, in which B⁻ denotes Cl⁻ or SO₄²⁻.

7. The method according to claim 6, in which PG represents BOC and B⁻ denotes Cl⁻.

8. The method according to claim 7, in which the base is a tertiary amine.

9. The method according to claim 8, in which the base is triethylamine (TEA), diisopropylethylamine (DIEA), N-methylmorpholine (NMM) or methylpiperidine.

10. The method according to claim 9, in which the (Z)-amino compound or the salt of the (Z)-amino compound and the compound of formula (II) react together in the same container in the presence of T3P and of the base.

11. The method according to claim 10, in which the acetonitrile/salts of the (Z)- and (E)-amino compounds proportion, expressed by weight, is between 5 and 17, preferably between 10 and 12.

12. The method according to claim 11, in which the temperature at which the enrichment is carried out is between 20 and 70° C.

13. The method according to claim 12, in which the benzyl alcohol/salts of the (Z)- and (E)-amino compounds proportion, expressed by weight, is between 1 and 4.

14. The use of T3P of formula (III): 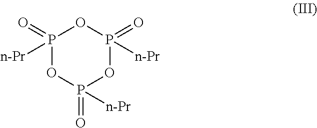
for coupling a (Z)-amino compound of formula 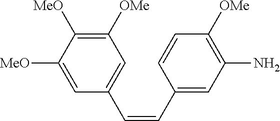
or the salt of the (Z)-amino compound of formula: 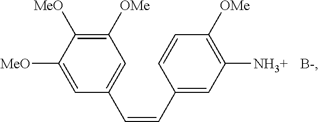
in which B⁻ denotes a counteranion,
with a doubly protected L-serine derivative of formula (II): 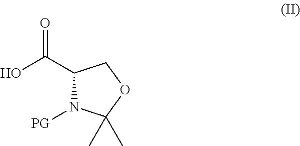
in which PG denotes a group protecting the amine function.

15. The use according to claim 14, in which PG denotes BOC, CBZ or FMOC and B⁻ denotes Cl⁻ or SO₄²⁻.

16. The use according to claim 15, in which the base is a tertiary amine.

17. The use according to claim 16, in which the (Z)-amino compound or the salt of the (Z)-amino compound and the compound of formula (II) react together in the same container in the presence of T3P and of the base.

18. The method according to claim 12, in which the benzyl alcohol/salts of the (Z)- and (E)-amino compounds proportion, expressed by weight, is between 2 and 3.
